(19) United States
(12) Patent Application Publication
(10) Pub. No.: US 2023/0260414 A1
(43) Pub. Date: Aug. 17, 2023

(54) DIGITAL COGNITION ENHANCEMENT TRAINING APPARATUS AND METHOD FOR COGNITIVE RESERVE ENHANCEMENT

(71) Applicant: HAII CORP., SEOUL (KR)

(72) Inventors: Junyoung LEE, Seoul (KR); Geonha KIM, Seoul (KR); Yoonyoung KANG, Gyeonggi-do (KR); Hyunjin CHO, Seoul (KR); Minkyoung SO, Seoul (KR); Museok KANG, Seoul (KR); Bo Hee KIM, Seoul (KR); Hanna CHUNG, Gyeonggi-do (KR)

(21) Appl. No.: 18/014,447

(22) PCT Filed: Oct. 22, 2021
(86) PCT No.: PCT/KR2021/014966

§ 371 Date: Jan. 4, 2023

(30) Foreign Application Priority Data
Oct. 22, 2020 (KR) 10-2020-0137792

(51) Int. Cl. G09B 5/02; G16H 20/70; G09B 19/00

(52) U.S. Cl. CPC G09B 5/02; G16H 20/70; G09B 19/00

(57) ABSTRACT

The present disclosure relates to a technology for effectively providing cognitive reinforcement training for cognitive reservability improvements in everyday life of a user, and supports the formation of exercise and eating habits which help improving the cognitive reservability of a user through an interactive program type system that is driven in a digital device, such as a smartphone, and allows the user to learn information on cognitive reinforcement training and a training method, and provides digitalized cognitive reinforcement training at a personalized difficulty level. Accordingly a user's training motive can be improved through a user-friendly format. Furthermore, the improvements of specialized and efficient cognitive reservability can be expected.

### TECHNICAL FIELD

This application is an application claiming priority to Korean Patent Application No. 10-2020-0137792 filed on Oct. 22, 2020, and all contents disclosed in the specification and drawings of the application are incorporated into this application by reference.

The present disclosure relates to a technology for effectively providing cognitive reinforcement training for cognitive reservability improvements in everyday life of a user, and more particularly, to providing an effect that is valid in reinforcing the cognitive reservability of a user by supporting the formation of exercise and eating habits which help improving the cognitive reservability of the user through an interactive program type system that is driven in a digital device, such as a smartphone, allowing the user to learn information on cognitive reinforcement training and a training method, and providing digitalized cognitive reinforcement training at a personalized difficulty level.

### BACKGROUND ART

Recently, interest is growing in dementia around the world. According to Ministry of Health and Welfare, dementia patients in Republic of Korea passed 700,000 in 2017, and dementia patients are expected to become 4.5 times in 2050.

In this case, dementia is a complex clinical syndrome in which the brain is damaged or destroyed due to a cause, such as an acquired trauma or a disease, and a cognitive function diminishes, and is one of typical diseases that are incurable through a conventional clinical treatment method.

Accordingly, it is very important to prevent dementia before dementia. For the prevention of dementia, a method of proactively managing dementia through cognitive reinforcement training, regular exercises above a certain intensity, and the management of vegetables, nuts, and fish-oriented diets from a mild cognitive impaired (MCI) stage, that is, a stage prior to dementia, and a normal stage is now emerging as the most efficient method of preventing dementia.

In this case, MCI means the state in which a cognitive function diminishes between normal aging and dementia. Since long-term non-pharmacological treatment rather than pharmacological treatment is effective in such an MCI, a cognitive reinforcement training program has been in the spotlight as a non-pharmacological treatment method.

Cognitive reinforcement training has an effect in preventing dementia by increasing cognitive reservability. The cognitive reservability is also the same concept as the immunity of the brain, and may be indirectly measured based on an education level, an activity level, etc. If the cognitive reservability is high, the time when dementia occurs may be delayed, and symptoms of development to dementia after its occurrence are reduced. Furthermore, related research revealed that the cognitive function of the aged to which cognitive intervention was provided was reinforced or maintained as the results of providing a complex cognitive reinforcement program, such as diet management and exercises, along with cognitive reinforcement training.

This disclosure is the result of carrying out the “Chatbot-based mental health smart healthcare system development for the elderly living alone” task(Task identification number: 1711122634, detailed task number: 2020M3C1B6112172) of the STEAM research (R&D) project of the 15 Ministry of Science and ICT from Nov. 2, 2020 to Jul. 31, 2021.

### DISCLOSURE

### Technical Problem

Various embodiments are directed to solving a problem which occurs when a cognitive reinforcement training program is provided as a non-pharmacological treatment method, that is, one of conventional cognitive disorder treatment methods, efficiently providing digitalized cognitive reinforcement training so that users can conveniently perform cognitive reinforcement training that helps improving cognitive reservability in their everyday life, and continuously managing the digitalized cognitive reinforcement training by reflecting a user' characteristics.

Most of the existing cognitive reinforcement training programs are provided at hospitals and counseling offices. In this case, there is a disadvantage in that a space movement and costs become a great burden to a patient. Furthermore, in the case of the existing computerized cognitive reinforcement training, it is difficult to continuously use the existing computerized cognitive reinforcement training because the learning of training and the installation of a program are difficult and complicated and the accessibility of a user and ease of use are low.

Accordingly, there is a need for a technology for a cognitive reinforcement training program that has been automatically customized suitably for a personal cognitive ability. Such a program may need to be provided in an environment in which the program is not a burden to a patient.

Furthermore, with the development of mobile communication, in many healthcare fields, training programs through digital media are being developed. In particular, in the case of a chronic disease such as dementia, there is an increasing need for a system capable of monitoring personal training around the clock and performing automated management.

### Technical Solution

In an embodiment, a digital cognitive reinforcement training apparatus for reinforcing cognitive reservability may include an interactive habit formation content provision unit configured to generate habit formation content including preemptive sentence content for providing mild cognitive impaired (MCI) and dementia-associated information and habit information that are collected through queries and answer sentence content suitable for context by analyzing text input by a user and to provide the generated habit formation content; a cognitive reinforcement training content provision unit configured to generate cognitive reinforcement training content including cognitive reinforcement training information for a plurality of cognitive domains and to provide the cognitive reinforcement training content to the user; a training result provision unit configured to calculate results of the training of the user for the provided training content and to provide the results of the training and a present training situation to the user; a training result feedback unit configured to derive training content difficulty level adjustment information and preferred training content information by analyzing the results of the training, to construct a personalized training content set so that an area that the user lacks of is able to be supplemented based on the derived information, and to incorporate the personalized training content set into next training of the user; and an interactive compensation provision unit configured to provide a reward to the user based on the results of the training and a training attendance rate and training completion rate of the user and to provide a personal memory remembrance question generated based on personal information of the user and text input by the user.

According to an embodiment of the present disclosure, user basic information may include at least one of a user name, a date of birth, gender, a user identification code, personal exercise habits, and personal eating habits. The interactive habit formation content provision unit may collect the basic information of the user in a query form.

According to an embodiment of the present disclosure, the interactive habit formation content provision unit may periodically provide the user with the habit formation content that is included in a habit list of exercise and eating habits that help a development of cognitive reservability, and may regularly provide a user task to the user based on habits selected by the user from the habit list.

According to an embodiment of the present disclosure, the interactive habit formation content provision unit may check whether the provided user task has been performed, may generate a compliment or warning sentence content when frequency of the execution is greater than or less than a preset reference range, and may provide the compliment or warning sentence content to the user.

According to an embodiment of the present disclosure, the cognitive reinforcement training content provision unit may provide the user with cognitive reinforcement training content having a game form, the execution of which needs to be completed within an allowed time every cycle that is input by the user or that is randomly preset, as a set unit.

According to an embodiment of the present disclosure, the cognitive reinforcement training content having the game form may include cognitive domain information that is a training target and a game tutorial. The cognitive reinforcement training content provision unit may provide the user with the cognitive domain information, the game tutorial, and game content that is preset within a limited time or randomly generated when the user performs a game.

According to an embodiment of the present disclosure, the cognitive reinforcement training content provision unit may randomly provide the cognitive reinforcement training content generated based on a plurality of cognitive domains, respectively, or may provide the cognitive reinforcement training content for an area that is preferred by the user or that requires intensive training, based on a selection of the user or the results of the training of the user.

According to an embodiment of the present disclosure, the training result feedback unit may collect the results of the training of the cognitive reinforcement training content performed by the user, may generate training result information including at least one of a training participation rate, a training achievement rate, results of training for each area by analyzing the collected results of the training, and may provide the training result information to the user.

According to an embodiment of the present disclosure, the cognitive reinforcement training content provision unit may include a memory training content generation unit configured to generate, in a game form, content including training to remind specific information; a language ability training content generation unit configured to generate, in a game form, content including association or analogy training performed by using a sentence or a word; an execution ability training content generation unit configured to generate, in a game form, training to determine the order of things over time; an attention concentration training content generation unit configured to generate, in a game form, content including training to concentrate attention; a calculation ability training content generation unit configured to generate, in a game form, training to improve a calculation ability by providing a four fundamental arithmetic operations problem; and a visual perception training content generation unit configured to generate, in a game form, training to improve a visual perception ability through training to expect a shape that is seen depending on a point of view of a specific object.

According to an embodiment of the present disclosure, the interactive compensation provision unit may provide the user with a reward having a stamp or point form by incorporating at least one of items including a training participation rate of the user, results of the execution of the habit formation content, and results of the execution of the cognitive reinforcement training, and may provide the user with present reward information of the provided reward.

According to an embodiment of the present disclosure, after providing the user with the present reward information, the interactive compensation provision unit may perform an induction of remembrance and a building of an alliance through a user personal item question including at least one of questions about thoughts, feelings, experiences, wishes, expectations, family matters, hobbies, religion, friendship, and his or her advantages and disadvantages.

According to an embodiment of the present disclosure, the memory training content generation unit may select one of games including “memorizing poems”, “memorizing national flags by country”, “memorizing local names”, a common sense quizzes, and “where to and what to eat” as the training to remind specific information, and may generate the selected game in the game form.

According to an embodiment of the present disclosure, the language ability training content generation unit may select one of games including “the four-character idiom”, “guess the first letter”, “the rearrangement of words”, and “please guess the name” as the association or analogy training that is performed by using the sentence or word, and may generate the selected game in the game form.

According to an embodiment of the present disclosure, the execution ability training content generation unit may select one of games including “let's eat”, “travel plans are fun”, and “ordering” as the training to determine the order of things over time, and may generate the selected game in the game form.

According to an embodiment of the present disclosure, the attention concentration training content generation unit may select one of “find words”, “find the same picture”, and a location movement game as the training to concentrate attention, and may generate the selected game in the game form.

According to an embodiment of the present disclosure, the calculation ability training content generation unit may select one of a receipt game, an “unlock the password” game, a subtract king game, and a “step by step from the basics” game as the training to improve the calculation ability by providing the four fundamental arithmetic operations problem, and may generate the selected game in the game form.

According to an embodiment of the present disclosure, the visual perception training content generation unit may select one of a shape prediction game and a “where was a picture taken” game that expects a figure of a shape that is seen depending on a view of a specific object, and may generate the selected game in the game form.

According to an embodiment of the present disclosure, the training result feedback unit may provide training having a different difficulty level for each user by individually adjusting the difficulty level based on the results of the training of the user.

According to an embodiment of the present disclosure, the training result feedback unit may provide a training game by raising the difficulty level of the cognitive reinforcement training content to be generated next to a higher level when the results of the training of the user are A, maintaining the difficulty level of the cognitive reinforcement training content to be generated next when the results of the training of the user is B, lowering the difficulty level of the cognitive reinforcement training content to be generated next to a lower level when the results of the training of the user is C, raising the difficulty level of the cognitive reinforcement training content to be generated next to a higher level when the results of the training of the user is a pass, and maintaining or lowering the difficulty level of the cognitive reinforcement training content to be generated next to a lower level when the results of the training of the user is a fail.

According to an embodiment of the present disclosure, the training result provision unit may include a personal information generation unit configured to provide user personal information including information on a name, gender, age, and training start date of the user; a present training situation information provision unit configured to provide present training situation information including a training attendance, the number of training absences, a training completion number, results of training for each area; and an area-based training result progress provision unit configured to provide a training progress based on results of the training up to now from a first training date of the user.

According to an embodiment of the present disclosure, the training result provision unit may periodically provide a registered third-party terminal with information on the results of the training of the user by using at least one of methods including SMS, a website, e-mail, and a messenger message with a consent of the user.

According to an embodiment of the present disclosure, the interactive habit formation content provision unit may identify a user cognitive state including an intention of the user based on text input by the user, may generate a natural query sentence suitable for the context as interactive habit formation content based on the identified user cognitive state, and may provide the natural query sentence to the user.

In another embodiment, a digital cognitive reinforcement training method for reinforcing cognitive reservability may include generating habit formation content including preemptive sentence content for providing mild cognitive impaired (MCI) and dementia-associated information and habit information that are collected through queries and answer sentence content suitable for context by analyzing text input by a user and providing the generated habit formation content; generating cognitive reinforcement training content including cognitive reinforcement training information for a plurality of cognitive domains and providing the cognitive reinforcement training content to the user; calculating results of the training of the user for the provided training content and providing the results of the training and a present training situation to the user; deriving training content difficulty level adjustment information and preferred training content information by analyzing the results of the training, constructing a personalized training content set so that an area that the user lacks of is able to be supplemented based on the derived information, and incorporating the personalized training content set into next training of the user; and providing a reward to the user based on the results of the training and a training attendance rate and training completion rate of the user and providing a personal memory remembrance question generated based on personal information of the user and text input by the user.

According to an embodiment of the present disclosure, the providing of the habit formation content may include periodically providing the user with the habit formation content that is included in a habit list of exercise and eating habits that help a development of cognitive reservability, and regularly providing a user task to the user based on habits selected by the user from the habit list.

According to an embodiment of the present disclosure, the providing of the cognitive reinforcement training content to the user may include generating, in a game form, content including training to remind specific information; generating, in a game form, content including association or analogy training performed by using a sentence or a word; generating, in a game form, training to determine the order of things over time; generating, in a game form, content including training to concentrate attention; generating, in a game form, training to improve a calculation ability by providing a four fundamental arithmetic operations problem; and generating, in a game form, training to improve a visual perception ability through training to expect a shape that is seen depending on a point of view of a specific object.

According to an embodiment of the present disclosure, the incorporating of the next training of the user may include providing a training game by raising the difficulty level of the cognitive reinforcement training content to be generated next to a higher level when the results of the training of the user is A, maintaining the difficulty level of the cognitive reinforcement training content to be generated next when the results of the training of the user is B, lowering the difficulty level of the cognitive reinforcement training content to be generated next to a lower level when the results of the training of the user is C, raising the difficulty level of the cognitive reinforcement training content to be generated next to a higher level when the results of the training of the user is a pass, and maintaining or lowering the difficulty level of the cognitive reinforcement training content to be generated next to a lower level when the results of the training of the user is a fail.

### Advantageous Effects

According to embodiments of the present disclosure, when cognitive reinforcement training is performed in a digital device, a user's training motive can be improved through a user-friendly format. Furthermore, the improvements of specialized and efficient cognitive reservability may be expected by assisting a cognitive reinforcement training effect in a way to provide a plurality of training games for 6 specialized cognitive domains, collect personal information of a user for exercise and eating habits, and provide the user with suitable habit formation content based on the collected personal information.

Furthermore, the continuity of training can be reinforced by applying the generation and management of personalized and systematic cognitive reinforcement training and habit formation to a messenger or an application of a digital device, such as a smartphone, providing the messenger or application to a user, allowing the user to learn a motive to continue such training in an interactive form, and periodically providing the motive.

Furthermore, the results of training collected through a user, user information, and a training difficulty level that is customized for a user and that is beyond the provision of simple training by combining the use information can be automatically provided. More positive and specialized cognitive reinforcement training can be provided by monitoring anomalies and a training progress situation of a user.

### DESCRIPTION OF DRAWINGS

FIG. 1 is a construction diagram of a digital cognitive reinforcement training apparatus for reinforcing cognitive reservability according to an embodiment of the present disclosure.

FIG. 2 is a detailed construction diagram of a cognitive reinforcement training content provision unit disclosed in FIG. 1.

FIG. 3 is a detailed construction diagram of a training result feedback unit disclosed in FIG. 1.

FIG. 4 is a diagram illustrating data sets for individually adjusting difficulty levels of cognitive reinforcement training content based on the results of the training of a user according to an embodiment of the present disclosure.

FIG. 5 illustrates a screen on which preemptive sentence content and habit formation content generated in a messenger platform according to an embodiment of the present disclosure are provided.

FIG. 6 illustrates a screen on which habit formation content including a habit list, which is generated in a messenger platform according to an embodiment of the present disclosure, is provided.

FIG. 7A, FIG. 7B, FIG. 7C and FIG. 7D illustrate screens on which a user is provided with attention concentration training content in a messenger platform according to an embodiment of the present disclosure.

FIG. 8A and FIG. 8B are diagrams illustrating that a response from a user is received by using a quick reply input method or a text input method in a messenger platform according to an embodiment of the present disclosure.

FIG. 9A, FIG. 9B, FIG. 9C, FIG. 9D, FIG. 9E and FIG. 9F illustrate screens on which calculation ability content, language ability content, attention content, execution ability content, memory content, and visual perception ability training content are provided in a messenger platform according to an embodiment of the present disclosure.

FIG. 10 is a screen illustrating that the results of training for training content provided in a messenger platform according to an embodiment of the present disclosure and a present training situation are provided.

FIG. 11 is a screen illustrating that a user reward and compliment sentence content are provided in a messenger platform according to an embodiment of the present disclosure.

FIG. 12 is a screen illustrating that personal information, present training situation information, and an area-based training result progress are provided in a messenger platform according to an embodiment of the present disclosure.

FIG. 13 is a flowchart of a digital cognitive reinforcement training method for reinforcing cognitive reservability according to an embodiment of the present disclosure.

### BEST MODE

Hereinafter, embodiments of the present disclosure are described in detail with reference to the accompanying drawings so that a person having ordinary knowledge in the art to which the present disclosure pertains may easily practice the embodiments. However, the present disclosure may be implemented in various different forms and is not limited to the embodiments described herein.

Furthermore, in the drawings, in order to clearly describe the present disclosure, a part not related to the description is omitted, and a similar reference number is used to refer to a similar part throughout the specification.

In the entire specification, when it is described that any part “includes” any element, this means that unless described otherwise, the any part may further include another element without excluding another element.

Hereinafter, a digital cognitive reinforcement training apparatus for reinforcing cognitive reservability according to an embodiment of the present disclosure and a method thereof are described with reference to the accompanying drawings.

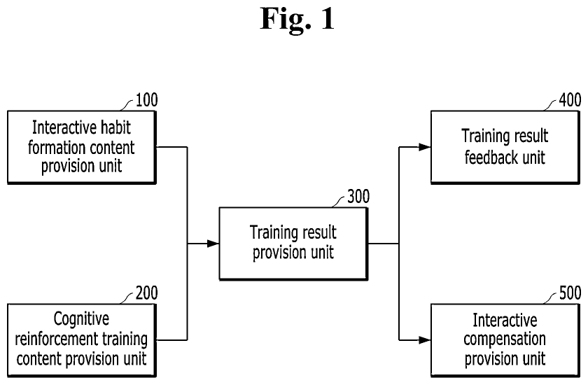

FIG. 1 is a construction diagram of a digital cognitive reinforcement training apparatus for reinforcing cognitive reservability according to an embodiment of the present disclosure.

Referring to FIG. 1, according to an embodiment of the present disclosure, the digital cognitive reinforcement training apparatus for reinforcing cognitive reservability may include an interactive habit formation content provision unit 100, a cognitive reinforcement training content provision unit 200, a training result provision unit 300, a training result feedback unit 400, and an interactive compensation provision unit 500.

According to an embodiment of the present disclosure, the digital cognitive reinforcement training apparatus for reinforcing cognitive reservability may be implemented on social network services (SNS) without a separate driving application, and may be driven based on a chatbot service.

In this case, the SNS may be KakaoTalk, LINE, WeChat, WhatsApp, Instagram, Facebook, etc., but is not limited thereto and may be used without limit in a platform capable of bi-directional communication with a user through a chatbot service.

The interactive habit formation content provision unit 100 may generate habit formation content, including preemptive sentence content for providing MCI and dementia-associated information and habit information that are collected through queries and answer sentence content suitable for context by analyzing text input by a user, and may provide the generated habit formation content.

In this case, the preemptive sentence may mean a sentence that is presented to the user through a system which is driven on a digital device so that the user may participate in training based on a preset time or cycle even in a situation in which the user has not taken any action. The preemptive sentence content may mean content including the preemptive sentence that is provided to the user.

According to an embodiment of the present disclosure, the preemptive sentence may be variously generated based on basic information of a user by using the preset sentence content generation model.

According to an embodiment of the present disclosure, the sentence content generation model may generate a dialogue corresponding to a response from a user by analyzing text input by the user.

According to an embodiment of the present disclosure, the text input by the user may mean text that is directly input by the user through the keyboard of a user terminal device, but is not limited thereto and may also include contents that are input in a quick reply format.

According to an embodiment of the present disclosure, the sentence content generation model may be a deep learning-based model, and may generate a sentence, including a more friendly word or expression that is frequently used by a user, by analyzing and learning a response from the user through deep learning.

According to an embodiment of the present disclosure, the sentence content generation model may generate a sentence which may become an answer or a re-question based on analysis information obtained by analyzing text input by a user. In this case, the accuracy of a sentence by the sentence content generation model can be improved by re-learning positive user responses.

According to an embodiment of the present disclosure, the preemptive sentence may be generated in a sentence format “000 (user), Hello. Good morning. Today is the 0-th day since 000 (user) started training. Please input “Start””, but is not limited thereto and may provide a variety of types of information, such as the elapse of a training progress, result information, and a different up to a goal as described above.

According to an embodiment of the present disclosure, basic information of a user includes at least one of a user name, a date of birth, gender, a user identification code, personal exercise habits, and personal eating habits. The interactive habit formation content provision unit 100 may collect the basic information of the user in a query form.

According to an embodiment of the present disclosure, the preemptive sentence including text capable of arousing a user's caution and enhancing a motive may be generated based on the basic information of a user.

According to an embodiment of the present disclosure, the interactive habit formation content provision unit 100 may periodically provide a user with habit formation content including a habit list of exercise or eating habits that help the development of cognitive reservability, and may regularly provide a user task based on habits selected from the habit list by the user.

According to an embodiment of the present disclosure, the habit list may include three aerobic exercises for 20 to 30 minutes or more a week, eating of whole grains three times a day, eating of green vegetables six times a week, eating of other vegetables once a day, eating of fish more than once a week, or eating of nuts five times or more a week.

According to an embodiment of the present disclosure, the interactive habit formation content provision unit 100 may check whether a provided user task has been performed, may generate a compliment or warning sentence content when frequency of the execution is greater than or less than a preset reference range, and may provide the compliment or warning sentence content to the user.

According to an embodiment of the present disclosure, the results of a task that has been actually performed by a user, among user tasks provided to the user, may be collected. When frequency of a task that has been performed by the user is greater than a preset reference, compliment sentence content may be generated and provided to the user in order to inspire the motivation of the user.

In contrast, when frequency of the task that has been performed by the user is less than the preset reference, warning sentence content may be generated and provided to the user in order to encourage the participation of the user in training.

According to an embodiment of the present disclosure, the interactive habit formation content provision unit 100 may identify a user cognitive state including the intention of a user based on text input by the user, may generate a natural query sentence suitable for context based on the identified user cognitive state, and may provide the user with the natural query sentence as interactive habit formation content.

According to an embodiment of the present disclosure, in order to identify the user cognitive state including the intention of a user based on text input by the user, the text that is directly input by the user may be received or the text may be received by using a quick reply input method.

According to an embodiment of the present disclosure, for older users who perform inaccurate inputs, the quick reply input method for more accurately recognize a user response compared to text may be used.

For example, if 1) an expression of a user, such as “I want to train”, “I do not want to train”, is important for the progress of a next dialogue or if 2) a user simply talks like “Aha” or “I see” based on a quick reply when an agent performs a long-winded conversation, a smooth interaction may be supported by presenting a selection passage.

According to an embodiment of the present disclosure, the meaning for each syllable may be identified by analyzing a response consisting of text that is directly input by a user. User cognitive state information including the intention of the user may be generated by collecting the identified meanings for each syllable.

According to the embodiment, in order to identify a meaning for each syllable by analyzing a response consisting of text that is directly input by a user, a syllable unit morpheme analysis method using a distribution of parts of speech and bi-directional LSTM CRFs may be used, but the present disclosure is not limited thereto.

According to an embodiment of the present disclosure, when a user response to a preemptive sentence consisting of text is received, a sentence that may naturally proceed to a next dialogue by duplicating the user response after the user response may be generated.

According to an embodiment of the present disclosure, natural language processing for the text analysis may be performed by using Dialogflow of Google.

The cognitive reinforcement training content provision unit 200 may generate cognitive reinforcement training content including cognitive reinforcement training information for a plurality of cognitive domains, and may provide the cognitive reinforcement training content to a user.

According to an embodiment of the present disclosure, the cognitive reinforcement training content provision unit 200 may generate cognitive reinforcement training content for at least one cognitive domain, among memory, the calculation ability, attention, the language ability, the execution ability, and visual perception, and may provide the cognitive reinforcement training content to a user.

According to an embodiment of the present disclosure, the cognitive reinforcement training content provision unit 200 may provide a user with cognitive reinforcement training content for each cognitive domain based on various criteria for each cycle, for each training progress degree, and for each training result.

According to an embodiment of the present disclosure, the cognitive reinforcement training content provision unit 200 may provide a user with cognitive reinforcement training content having a game form, the execution of which needs to be completed within an allowed time every cycle that is input by the user or that is randomly preset, as a set unit.

According to an embodiment of the present disclosure, in providing cognitive reinforcement training content, the quick reply input method or a pressable form, such as an icon, may be used, but the present disclosure is not limited thereto. A sentence that recommends a cognitive reinforcement treatment training item may be generated by using the sentence content generation model, and may be presented in an interactive form.

According to an embodiment of the present disclosure, cognitive reinforcement training content having a game form includes cognitive domain information, that is, a training target, and a game tutorial. The cognitive reinforcement training content provision unit 200 may provide a user with the cognitive domain information, the game tutorial, and game content that is preset within a limited time or that is randomly generated when the user plays a game.

In this case, the cognitive domain information may mean information that is arranged and provided so that the user can easily understand the cognitive domain on which training will be performed. The game tutorial may mean all types of information that are provided so that a user can previously experience and learn a method of performing cognitive reinforcement training content that will be provided to the user in the same form as that of a game to be provided and a direction in which the method is performed.

According to an embodiment of the present disclosure, the cognitive reinforcement training content provision unit 200 may randomly provide pieces of cognitive reinforcement training content that are generated based on a plurality of cognitive domains, respectively, or may provide cognitive reinforcement training content for an area that is preferred by a user or that requires intensive training based on the selection of the user or the results of the training of the user.

According to an embodiment of the present disclosure, the cognitive reinforcement training content may be generated for each cognitive domain based on a preset criterion, and may be generated for each stage or for each training date.

The training result provision unit 300 may calculate the results of the training of a user for provided training content, and may provide the user with the results of the training and a present training situation.

According to an embodiment of the present disclosure, the results of the training of a user may be calculated by analyzing information that is collected in a process of performing cognitive reinforcement training content provided to the user, and may be provided to the user. A present training situation that is generated by periodically synthesizing the results of the training may also be provided to the user.

According to an embodiment of the present disclosure, the training result provision unit 300 may collect the results of training for cognitive reinforcement training content that has been performed by a user, may generate training result information including at least one of a training participation rate, a training achievement rate, the results of training for each area by analyzing the collected results of the training, and may provide the training result information to the user.

In this case, the training participation rate may mean information on frequency of cognitive reinforcement training that has been performed by the user for each cycle. The training achievement rate may mean information on a degree of progress or a mark of the cognitive reinforcement training content that has been performed. The results of the training for each area may mean information in which marks of cognitive reinforcement training performed for each cognitive domain have been integrated.

According to an embodiment of the present disclosure, the training result provision unit 300 may periodically provide a registered third-party terminal with information on the results of the training of a user by using at least one of methods including SMS, a website, e-mail, and a messenger message, with the consent of the user.

The training result feedback unit 400 may derive training content difficulty level adjustment information and preferred training content information by analyzing the results of the training of a user, may construct a personalized training content set so that an area that the user lacks of can be supplemented, based on the derived information, and may incorporate the personalized training content set into next training of the user.

According to an embodiment of the present disclosure, the personalized training content set may mean a content set consisting of at least one piece of cognitive reinforcement training content for a cognitive domain having low training results based on the results of the training of a user so that the user can be more intensively trained with the corresponding cognitive domain.

According to an embodiment of the present disclosure, the training result feedback unit 400 may individually adjust a difficulty level based on the results of the training of a user, and may provide training having a different difficulty level for each user.

According to an embodiment of the present disclosure, the training result feedback unit 400 may analyze the results of training, may generate a difficulty level by raising the difficulty level when generating next training content when the results of the training are relatively high based on the analyzed results, and may generate a difficulty level by lowering the difficulty level when generating next training content when the results of the training are relatively low based on the analyzed results.

According to an embodiment of the present disclosure, the training result feedback unit 400 may provide a training game by raising the difficulty level of cognitive reinforcement training content to be generated next to a higher level when the results of the training of a user is A, maintaining the difficulty level of cognitive reinforcement training content to be generated next when the results of the training of a user is B, lowering the difficulty level of cognitive reinforcement training content to be generated next to a lower level when the results of the training of a user is C, raising the difficulty level of cognitive reinforcement training content to be generated next to a higher level when the results of the training of a user is a pass, and maintaining or lowering the difficulty level of cognitive reinforcement training content to be generated next to a lower level when the results of the training of a user is a fail.

The interactive compensation provision unit 500 may provide a reward to a user based on the results of the training of a user and a training attendance rate and training completion rate of the user, and may provide a personal memory remembrance question that is generated based on user personal information and text input by the user.

According to an embodiment of the present disclosure, the reward may mean compensation capable of inspiring the motivation of a user, such as a point, a stamp, appellation, or a gift, when a training number, a training participation rate, a training achievement rate, or a training mark is equal to or greater than a predetermined reference, but any reward may be used without limit if the reward can be accepted by a user as a compensatory meaning.

According to an embodiment of the present disclosure, the personal memory remembrance question may mean a question about a personal item for the remembrance of memory and the establishment of ties for a user, and may include a user personal item question.

According to an embodiment of the present disclosure, after providing the user with the present reward information, the interactive compensation provision unit 500 may perform the induction of remembrance and the building of an alliance through a user personal item question including at least one of questions about thoughts, feelings, experiences, wishes, expectations, family matters, hobbies, religion, friendship, and his or her advantages and disadvantages.

According to an embodiment of the present disclosure, in order to perform the induction of remembrance and the building of an alliance, at least one of questions about thoughts, feelings, experiences, wishes, expectations, family matters, hobbies, religion, friendship, and his or her advantages and disadvantages may be included in a personal item question. Question items are not limited to the above examples.

According to an embodiment of the present disclosure, the interactive compensation provision unit 500 may provide a user with a reward having a stamp or point form by incorporating at least one of items, including a training participation rate of the user, the results of execution of habit formation content of the user, and the results of execution of cognitive reinforcement training of the user, and may provide present reward information of the provided reward.

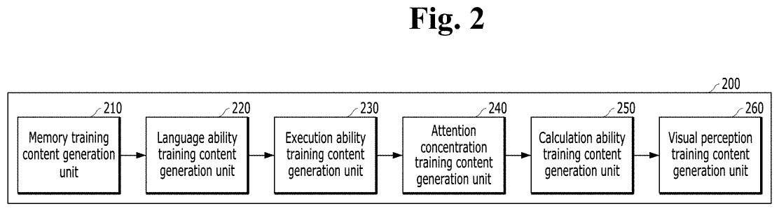

FIG. 2 is a detailed construction diagram of a cognitive reinforcement training content provision unit 200 disclosed in FIG. 1.

Referring to FIG. 2, the cognitive reinforcement training content provision unit 200 according to an embodiment of the present disclosure may include a memory training content generation unit 210, a language ability training content generation unit 220, an execution ability training content generation unit 230, an attention concentration training content generation unit 240, a calculation ability training content generation unit 250, and a visual perception training content generation unit 260.

The memory training content generation unit 210 may generate content including training to remind specific information in a game form.

According to an embodiment of the present disclosure, the memory training content generation unit 210 may select one of games, such as “memorizing poems”, “memorizing national flags by country”, “memorizing local names”, a common sense quizzes, and “where to and what to eat”, as training to remind specific information, and may generate the selected game in a game form.

The language ability training content generation unit 220 may generate content including association or analogy training that is performed by using a sentence or a word in a game form.

According to an embodiment of the present disclosure, the language ability training content generation unit 220 may select one of games, such as “a four-character idiom”, “guess the first letter”, “the rearrangement of words”, and “please guess the name”, as association or analogy training that is performed by using a sentence or a word, and may generate the selected game in a game form.

The execution ability training content generation unit 230 may generate training to determine the order of things over time in a game form.

According to an embodiment of the present disclosure, the execution ability training content generation unit 230 may select one of games, such as “let's eat”, “travel plans are fun”, and “ordering”, as training to determine the order of things over time, and may generate the selected game in a game form.

The attention concentration training content generation unit 240 may generate content including training to concentrate attention in a game form.

According to an embodiment of the present disclosure, the attention concentration training content generation unit 240 may select one of games, such as “find words”, “find the same picture”, and a location movement, as training to concentrate attention, and may generate the selected game in a game form.

The calculation ability training content generation unit 250 may generate training to improve the calculation ability by providing the four fundamental arithmetic operations problem in a game form.

According to an embodiment of the present disclosure, the calculation ability training content generation unit 250 may select one of a receipt game, an “unlock the password” game, a subtract king game, and a “step by step from the basics” game as training to improve the calculation ability by providing the four fundamental arithmetic operations problem, and may generate the selected game in a game form.

The visual perception training content generation unit 260 may generate training to improve the visual perception ability through training to expect a shape that is seen depending on a point of view of a specific object in a game form.

According to an embodiment of the present disclosure, the visual perception training content generation unit 260 may select one of a shape prediction game and a “where was the picture taken” game that expects a figure of a shape that is seen depending on a view of a specific object, and may generate the selected game in a game form.

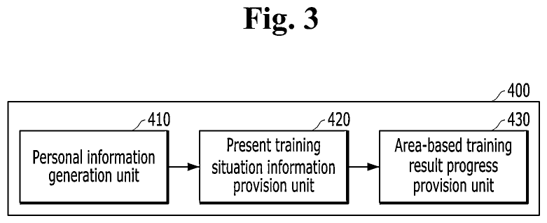

FIG. 3 is a detailed construction diagram of the training result feedback unit 400 disclosed in FIG. 1.

Referring to FIG. 3, the training result feedback unit 400 according to an embodiment of the present disclosure may include a personal information generation unit 410, a present training situation information provision unit 420, and an area-based training result progress provision unit 430.

The personal information generation unit 410 may provide user personal information including information on the name, gender, age, and training start date of a user.

According to an embodiment of the present disclosure, the user personal information may be information generated by using information on the name, gender, age, and training start date of a user that are received from the user, but personal information of a user that is necessary for training may be used without limit.

The present training situation information provision unit 420 may provide present training situation information including a training attendance, the number of training absences, a training completion number, and the results of training for each area.

According to an embodiment of the present disclosure, the present training situation information may mean information that has been arranged in a time series by collecting the results of training that has been performed by a user. Any data that has been arranged in a time series with respect to a specific training item, in addition to the training attendance, the number of training absences, the training completion number, and the results of training for each area, may be used as the present training situation information without limit.

The area-based training result progress provision unit 430 may provide a training progress based on the results of training up to now from the first training date of a user.

According to an embodiment of the present disclosure, the training progress may be provided to a user in a graph form, but the present disclosure is not limited thereto. Any form that may show a time-series progress may be used without limit.

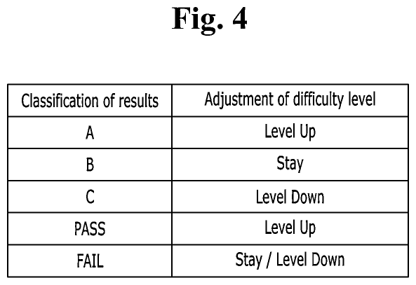

FIG. 4 is a diagram illustrating data sets for individually adjusting a difficulty level of cognitive reinforcement training content based on the results of the training of a user according to an embodiment of the present disclosure.

FIG. 4 illustrates data sets for adjusting difficulty levels of training content according to an embodiment of the present disclosure.

According to an embodiment of the present disclosure, difficulty levels may be adjusted based on the results of training that has been performed by a user based on cognitive reinforcement training content. The results of the training may be calculated as A, B, C, P, or F. Accordingly, the difficulty level may be raised, maintained, or lowered.

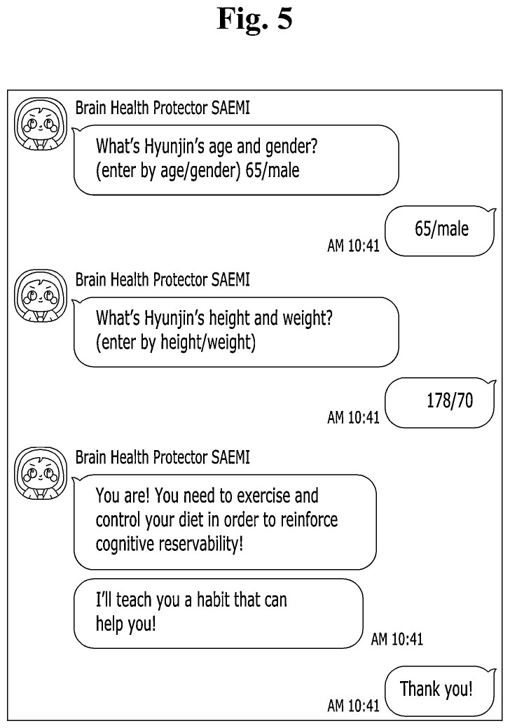

FIG. 5 illustrates a screen on which preemptive sentence content and habit formation content generated in a messenger platform according to an embodiment of the present disclosure are provided.

FIG. 5 illustrates a screen that provides a user with the preemptive sentence content and the habit formation content in a messenger platform.

According to an embodiment of the present disclosure, as in FIG. 5, sentences to ask questions about user personal information, such as the age, sex, height, and weight of a user, may be presented to the user as the preemptive sentence content. The habit formation content including answer sentence content to be provided to the user for the formation of habits may be provided based on answers of the user to the questions.

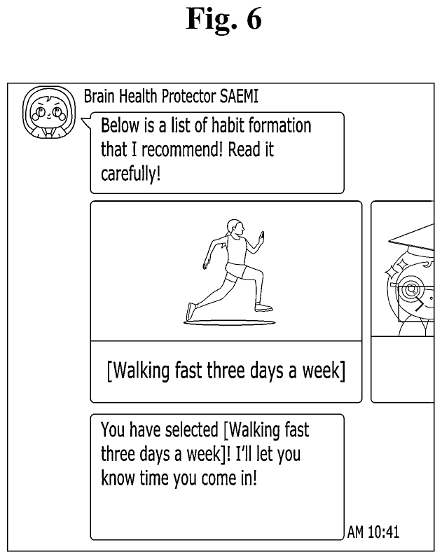

FIG. 6 illustrates a screen on which habit formation content including a habit list, which is generated in a messenger platform according to an embodiment of the present disclosure, is provided.

FIG. 6 illustrates the habit formation content including the habit list that is provided to a user.

According to an embodiment of the present disclosure, as in FIG. 6, the habit formation content for providing MCI and dementia-associated information and habit information may be provided by analyzing text input by the user. In particular, a good habit list may be generated for cognitive reinforcement. As in FIG. 6, habit information included in the habit list and a user task (“3-day power walking a week”) based on the provided habit information may be provided.

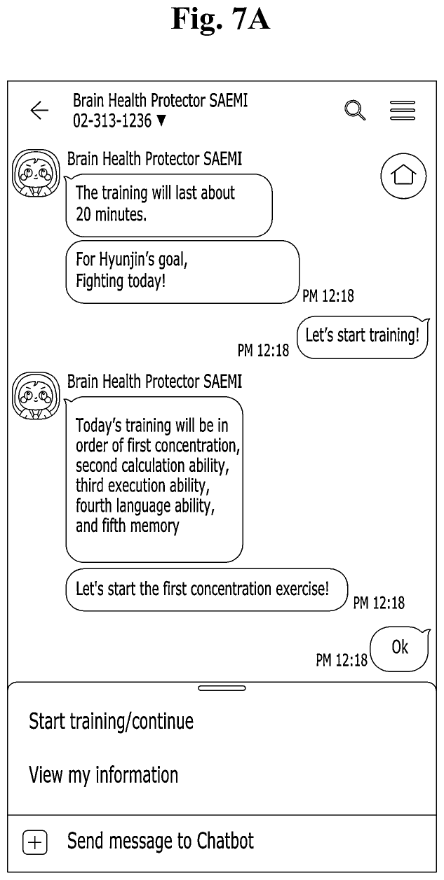

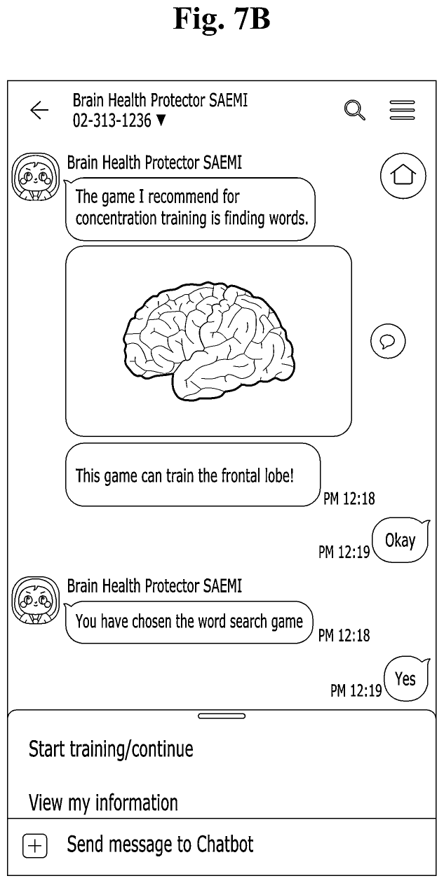

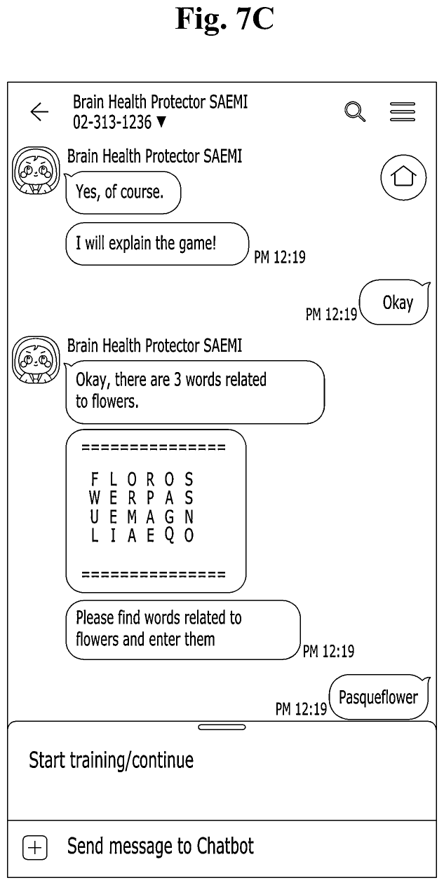

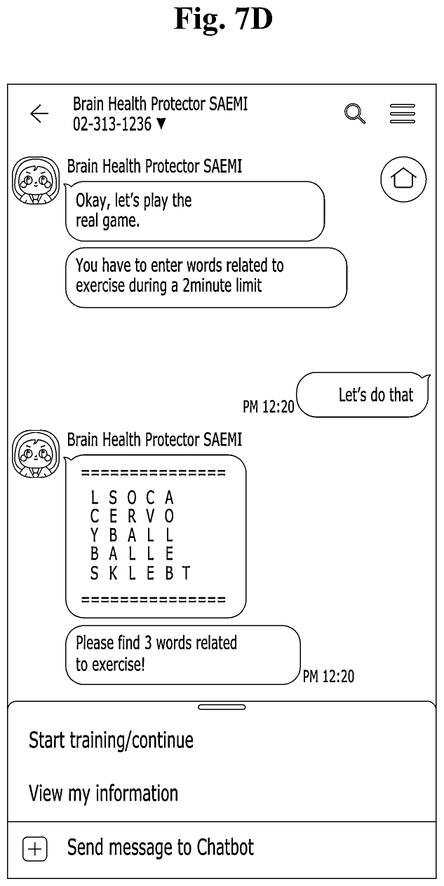

FIG. 7A, FIG. 7B, FIG. 7C and FIG. 7D illustrate screens on which a user is provided with attention concentration training content in a messenger platform according to an embodiment of the present disclosure.

FIG. 7A, FIG. 7B, FIG. 7C and FIG. 7D illustrate screens on which attention concentration training content according to an embodiment of the present disclosure is provided to a user. The start of cognitive reinforcement training may be suggested through preemptive sentence content, items of cognitive reinforcement training for each cognitive domain may be presented with the consent of a user. Cognitive reinforcement training content for a specific cognitive domain that is selected based on a user input or a recommendation may be provided.

According to an embodiment of the present disclosure, as in FIG. 7A, FIG. 7B, FIG. 7C and FIG. 7D, cognitive domain information (e.g., a description of frontal lobe training), that is, a training target, and a game tutorial may be provided. When the game tutorial is completed, real game content may be provided to the user.

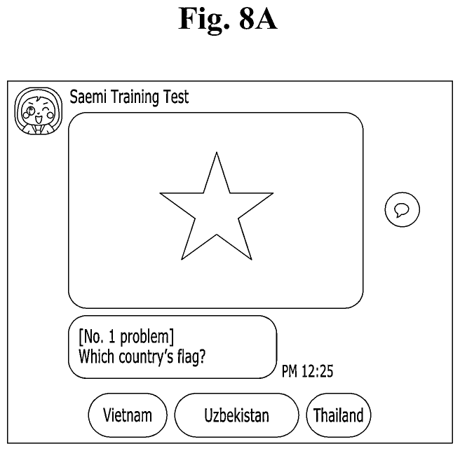

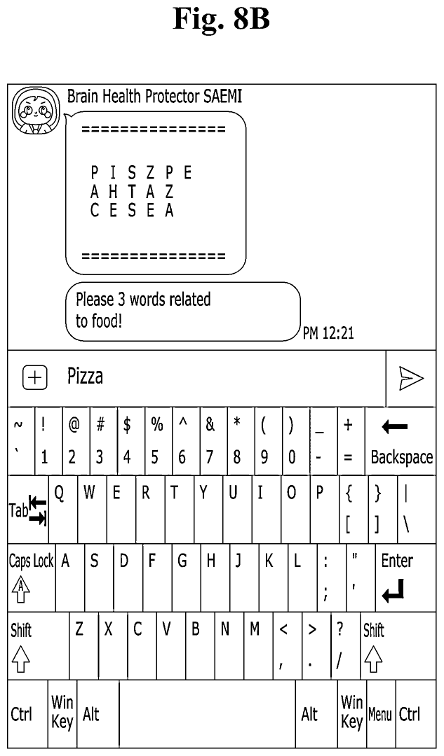

FIG. 8A and FIG. 8B are diagrams illustrating that a response from a user is received by using a quick reply input method or a text input method in a messenger platform according to an embodiment of the present disclosure.

FIG. 8A and FIG. 8B illustrate the two methods of inputting, by a user, an answer to cognitive reinforcement training content provided to the user in a process of the user performing the cognitive reinforcement training content according to an embodiment of the present disclosure.

According to an embodiment of the present disclosure, as in a screen of FIG. 8A, a user may input an answer by using the quick reply input method which has a low degree of freedom, but can reduce an erroneous input probability. As in a screen of FIG. 8B, a user may input an answer by using the text input method through a keyboard which has a high erroneous input probability, but guarantee a high degree of freedom.

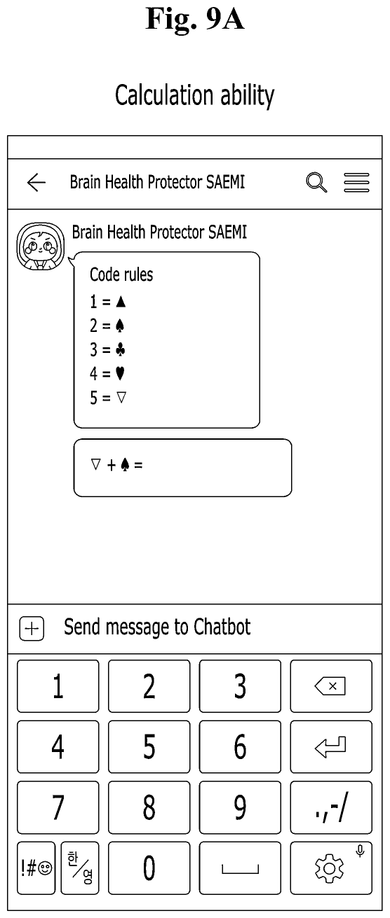

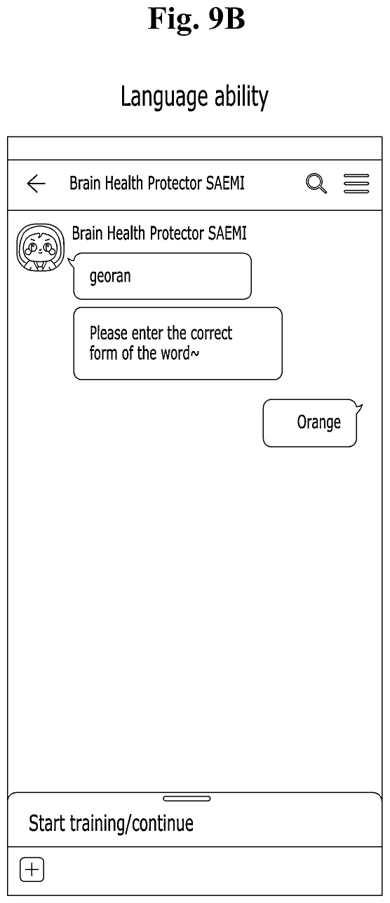

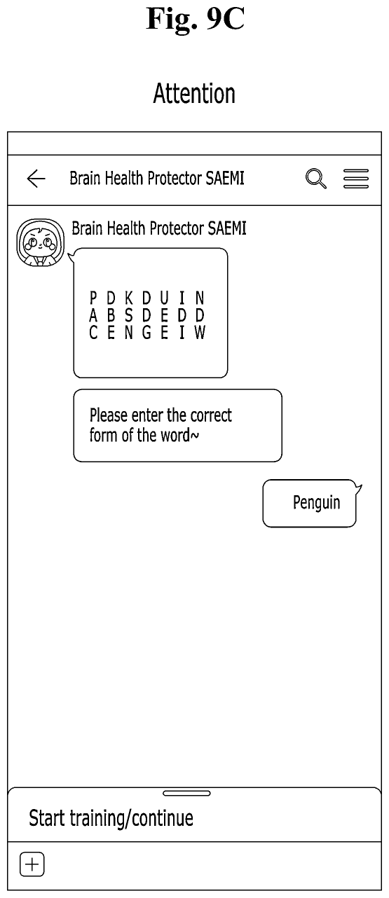

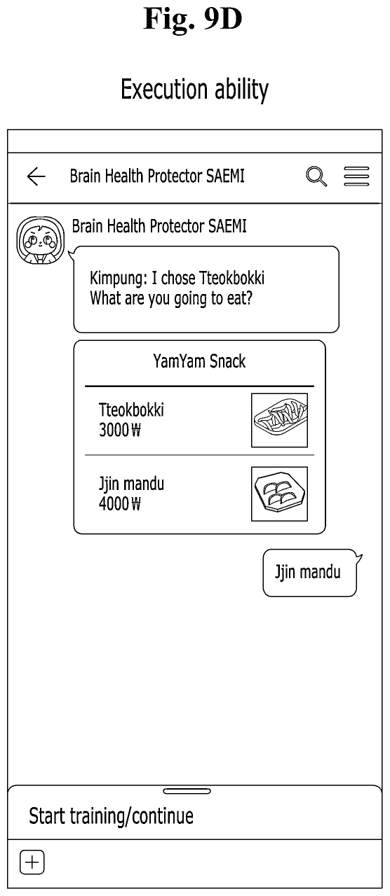

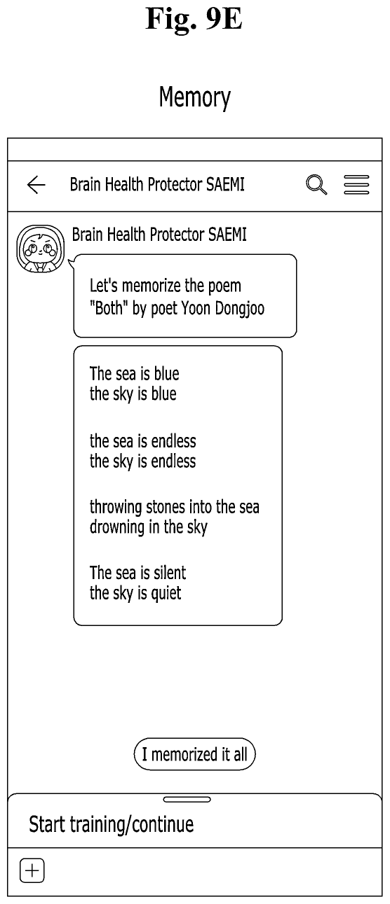

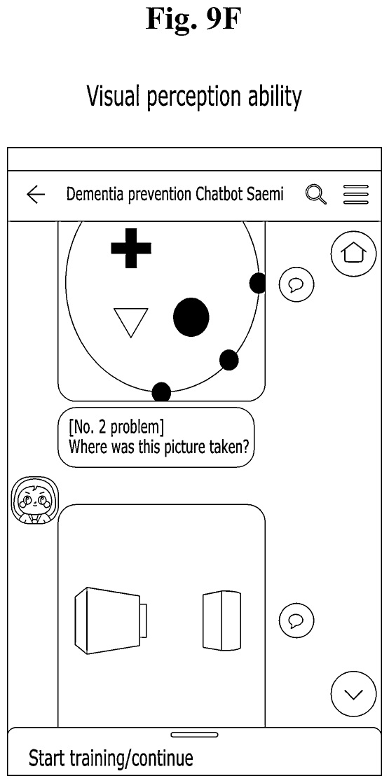

FIG. 9A, FIG. 9B, FIG. 9C, FIG. 9D, FIG. 9E and FIG. 9F are screens illustrating that calculation ability content, language ability content, attention content, execution ability content, memory content, and visual perception ability training content are provided in a messenger platform according to an embodiment of the present disclosure.

FIG. 9A, FIG. 9B, FIG. 9C, FIG. 9D, FIG. 9E and FIG. 9F illustrate screens on which cognitive reinforcement training content for each cognitive domain according to an embodiment of the present disclosure is provided.

As in FIG. 9A, FIG. 9B, FIG. 9C, FIG. 9D, FIG. 9E and FIG. 9F, unique cognitive reinforcement training content capable of reinforcing a corresponding cognitive domain for each cognitive domain, such as the calculation ability, the language ability, attention, the execution ability, memory, and the visual perception ability may be provided.

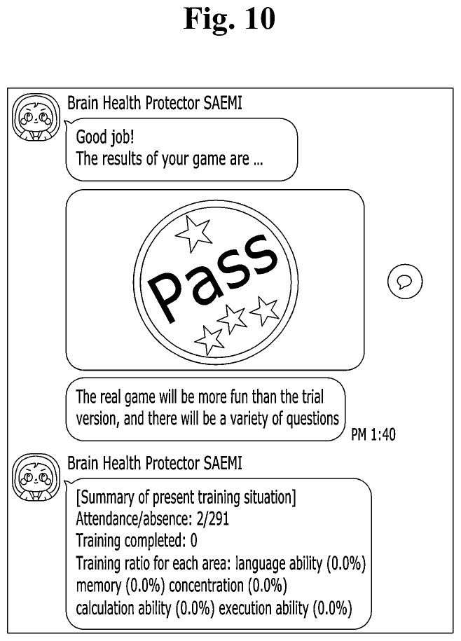

FIG. 10 illustrates a screen on which the results of training for training content provided in a messenger platform according to an embodiment of the present disclosure and a present training situation are provided.

FIG. 10 illustrates a screen on which the results of training for training content that has been performed by a user and a present training situation are provided in a messenger platform according to an embodiment of the present disclosure.

According to an embodiment of the present disclosure, as in FIG. 10, when a user completes the execution of cognitive reinforcement training content, the results of training for the executed training content may be provided to the user. A present training situation including an attendance/the number of absences, a training completion number, and a training ratio for each area may be provided to the user by analyzing the results of training that has already been performed in a time series.

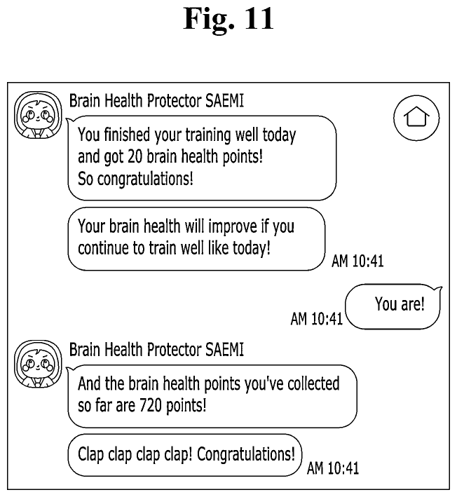

FIG. 11 is a screen illustrating that a user reward and compliment sentence content are provided in a messenger platform according to an embodiment of the present disclosure.

According to an embodiment of the present disclosure, when a user completes training based on cognitive reinforcement training content, as in FIG. 11, a reward (“20 points”) may be provided to the user based on a training attendance rate and training completion rate of the user. When accumulated reward information, a predetermined number of rewards, or an accumulated reward is achieved, compliment sentence content may be generated and provided to the user.

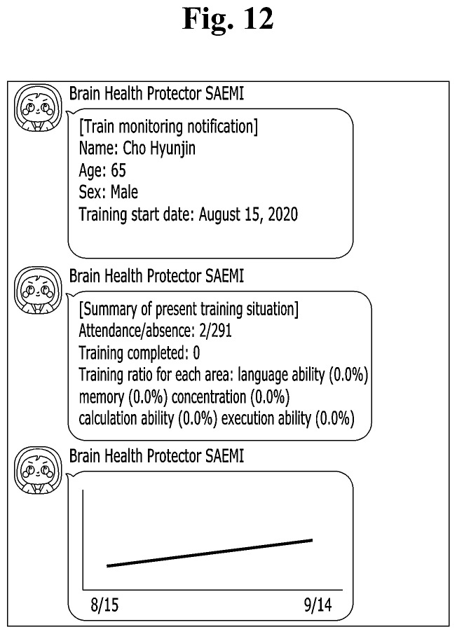

FIG. 12 is a screen illustrating that personal information, present training situation information, and an area-based training result progress are provided in a messenger platform according to an embodiment of the present disclosure.

According to an embodiment of the present disclosure, as in FIG. 12, personal information, present training situation information, and a training result progress for each area may be provided in the messenger platform. In particular, the progress of the results of training for each area may be provided in a graph form.

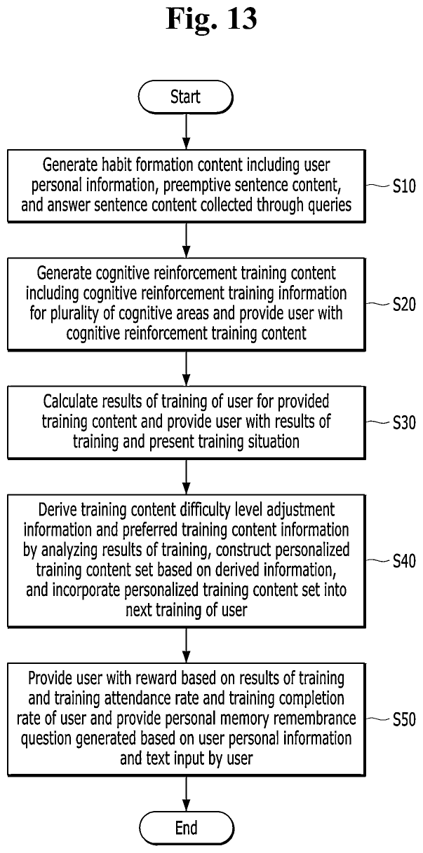

FIG. 13 is a flowchart of a digital cognitive reinforcement training method for reinforcing cognitive reservability according to an embodiment of the present disclosure.

Habit formation content including user personal information, preemptive sentence content, and answer sentence content that are collected through queries is generated. The habit formation content is provided (S10).

According to an embodiment of the present disclosure, habit formation content, including preemptive sentence content for providing MCI and dementia-associated information and habit information that are collected through queries and answer sentence content suitable for context by analyzing text input by a user, may be generated. The generated habit formation content may be provided.

According to an embodiment of the present disclosure, the preemptive sentence may be variously generated based on basic information of a user by using the preset sentence content generation model.

According to an embodiment of the present disclosure, the sentence content generation model may generate a dialogue corresponding to a response from a user by analyzing text input by the user.

According to an embodiment of the present disclosure, the text input by the user may mean text that is directly input by the user through the keyboard of a user terminal device, but is not limited thereto and may also include contents that are input in a quick reply format.

According to an embodiment of the present disclosure, the sentence content generation model may be a deep learning-based model, and may generate a sentence, including a more friendly word or expression that is frequently used by a user, by analyzing and learning a response from the user through deep learning.

According to an embodiment of the present disclosure, the sentence content generation model may generate a sentence which may become an answer or a re-question based on analysis information obtained by analyzing text input by a user. In this case, the accuracy of a sentence by the sentence content generation model can be improved by re-learning positive user responses.

According to an embodiment of the present disclosure, the preemptive sentence may be generated in a sentence format “000 (user), Hello. Good morning. Today is the 0-th day since 000 (user) started training. Please input “Start””, but is not limited thereto and may provide a variety of types of information, such as the elapse of a training progress, result information, and a different up to a goal as described above.

According to an embodiment of the present disclosure, the basic information of a user may use at least one of a user name, a date of birth, gender, a user identification code, personal exercise habits, and personal eating habits. The basic information of the user may be collected in a query form.

According to an embodiment of the present disclosure, the preemptive sentence including text capable of arousing a user's caution and enhancing a motive may be generated based on the basic information of a user.

According to an embodiment of the present disclosure, habit formation content may include a habit list of exercise or eating habits that help the development of cognitive reservability, and may be periodically provided to a user. A user task may be regularly provided based on habits selected from the habit list by the user.

According to an embodiment of the present disclosure, whether the provided user task has been performed may be checked. When frequency of the execution is greater than or less than a preset reference range, a compliment or warning sentence content may be generated and provided to a user.

According to an embodiment of the present disclosure, the results of a task that has been actually performed by a user, among user tasks provided to the user, may be collected. When frequency of a task that has been performed by the user is greater than a preset reference, compliment sentence content may be generated and provided to the user in order to inspire the motivation of the user.

In contrast, when frequency of the task that has been performed by the user is less than the preset reference, warning sentence content may be generated and provided to the user in order to encourage the participation of the user in training.

According to an embodiment of the present disclosure, a user cognitive state including the intention of a user may be identified based on text input by the user, and a natural query sentence suitable for context may be generated based on the identified user cognitive state and provided to the user as interactive habit formation content.

According to an embodiment of the present disclosure, in order to identify the user cognitive state including the intention of a user based on text input by the user, the text that is directly input by the user may be received or the text may be received by using a quick reply input method.

According to an embodiment of the present disclosure, for older users who perform inaccurate inputs, the quick reply input method for more accurately recognize a user response compared to text may be used.

According to an embodiment of the present disclosure, the meaning for each syllable may be identified by analyzing a response consisting of text that is directly input by a user. User cognitive state information including the intention of the user may be generated by collecting the identified meanings for each syllable.

According to the embodiment, in order to identify a meaning for each syllable by analyzing a response consisting of text that is directly input by a user, a syllable unit morpheme analysis method using a distribution of parts of speech and bi-directional LSTM CRFs may be used, but the present disclosure is not limited thereto.

According to an embodiment of the present disclosure, when a user response to a preemptive sentence consisting of text is received, a sentence that may naturally proceed to a next dialogue by duplicating the user response after the user response may be generated.

According to an embodiment of the present disclosure, natural language processing for the text analysis may be performed by using Dialogflow of Google.

Cognitive reinforcement training content including cognitive reinforcement training information for a plurality of cognitive domains is generated, and is provided to the user (S20).

According to an embodiment of the present disclosure, cognitive reinforcement training content including cognitive reinforcement training information for a plurality of cognitive domains may be generated and provided to a user.

According to an embodiment of the present disclosure, cognitive reinforcement training content for at least one cognitive domain, among memory, the calculation ability, attention, the language ability, the execution ability, and visual perception, may be generated and provided to a user.

According to an embodiment of the present disclosure, cognitive reinforcement training content for each cognitive domain may be provided to a user based on various criteria for each cycle, for each training progress degree, and for each training result.

According to an embodiment of the present disclosure, cognitive reinforcement training content having a game form, the execution of which needs to be completed within an allowed time every cycle that is input by a user or that is randomly preset, may be provided to the user as a set unit.

According to an embodiment of the present disclosure, in providing cognitive reinforcement training content, the quick reply input method or a pressable form, such as an icon, may be used, but the present disclosure is not limited thereto. A sentence that recommends a cognitive reinforcement treatment training item may be generated by using the sentence content generation model, and may be presented in an interactive form.

According to an embodiment of the present disclosure, cognitive reinforcement training content having a game form may include cognitive domain information, that is, a training target, and a game tutorial. The cognitive domain information, the game tutorial, and game content that is preset within a limited time or that is randomly generated when a user plays a game may be provided to the user.

In this case, the cognitive domain information may mean information that is arranged and provided so that the user can easily understand the cognitive domain on which training will be performed. The game tutorial may mean all types of information that are provided so that a user can previously experience and learn a method of performing cognitive reinforcement training content that will be provided to the user in the same form as that of a game to be provided and a direction in which the method is performed.

According to an embodiment of the present disclosure, pieces of cognitive reinforcement training content that are generated based on a plurality of cognitive domains, respectively, may be randomly provided, or cognitive reinforcement training content for an area that is preferred by a user or that requires intensive training may be provided based on the selection of the user or the results of the training of the user.

According to an embodiment of the present disclosure, the cognitive reinforcement training content may be generated for each cognitive domain based on a preset criterion, and may be generated for each stage or for each training date.

According to an embodiment of the present disclosure, content including training to remind specific information may be generated in a game form.

According to an embodiment of the present disclosure, one of games, such as “memorizing poems”, “memorizing national flags by country”, “memorizing local names”, a common sense quizzes, and “where to and what to eat”, may be selected as training to remind specific information, and may be generated in a game form.

According to an embodiment of the present disclosure, content including association or analogy training that is performed by using a sentence or a word may be generated in a game form.

According to an embodiment of the present disclosure, one of games, such as “a four-character idiom”, “guess the first letter”, “the rearrangement of words”, and “please guess the name”, may be selected as association or analogy training that is performed by using a sentence or a word, and may be generated in a game form.

According to an embodiment of the present disclosure, training to determine the order of things over time may be generated in a game form.

According to an embodiment of the present disclosure, one of games, such as “let's eat”, “travel plans are fun”, and “ordering”, may be selected as training to determine the order of things over time, and may be generated in a game form.

According to an embodiment of the present disclosure, content including training to concentrate attention may be generated in a game form.

According to an embodiment of the present disclosure, one of games, such as “find words”, “find the same picture”, and a location movement, may be selected as training to concentrate attention, and may be generated in a game form.

According to an embodiment of the present disclosure, training to improve the calculation ability by providing the four fundamental arithmetic operations problem may be generated in a game form.

According to an embodiment of the present disclosure, one of a receipt game, an “unlock the password” game, a subtract king game, and a “step by step from the basics” game may be selected as training to improve the calculation ability by providing the four fundamental arithmetic operations problem, and may be generated in a game form.

According to an embodiment of the present disclosure, training to improve the visual perception ability through training to expect a shape that is seen depending on a point of view of a specific object may be generated in a game form.

According to an embodiment of the present disclosure, one of a shape prediction game and a “where was the picture taken” game that expects a figure of a shape that is seen depending on a view of a specific object may be selected and generated in a game form.

The results of the training of the user for the provided training content are calculated, and the results of the training and a present training situation are provided to the user (S30).

According to an embodiment of the present disclosure, the results of the training of a user for provided training content may be calculated, and the results of the training and a present training situation may be provided to the user.

According to an embodiment of the present disclosure, the results of the training of a user may be calculated by analyzing information that is collected in a process of performing cognitive reinforcement training content provided to the user, and may be provided to the user. A present training situation that is generated by periodically synthesizing the results of the training may also be provided to the user.

According to an embodiment of the present disclosure, the results of training for cognitive reinforcement training content that has been performed by a user may be collected, training result information including at least one of a training participation rate, a training achievement rate, the results of training for each area may be generated by analyzing the collected results of the training, and the training result information may be provided to the user.

According to an embodiment of the present disclosure, the training participation rate may mean information on frequency of cognitive reinforcement training that has been performed by the user for each cycle. The training achievement rate may mean information on a degree of progress or a mark of the cognitive reinforcement training content that has been performed. The results of the training for each area may mean information in which marks of cognitive reinforcement training performed for each cognitive domain have been integrated.

According to an embodiment of the present disclosure, information on the results of the training of a user may be periodically provided to a registered third-party terminal by using at least one of methods including SMS, a website, e-mail, and a messenger message, with the consent of the user.

Training content difficulty level adjustment information and preferred training content information are derived by analyzing the results of the training. A personalized training content set is constructed based on the derived information and incorporated into next training of the user (S40).

According to an embodiment of the present disclosure, training content difficulty level adjustment information and preferred training content information may be derived by analyzing the results of the training of a user. A personalized training content set may be constructed so that an area that the user lacks of can be supplemented, based on the derived information, and may be incorporated into next training of the user.

According to an embodiment of the present disclosure, the personalized training content set may mean a content set consisting of at least one piece of cognitive reinforcement training content for a cognitive domain having low training results based on the results of the training of a user so that the user can be more intensively trained with the corresponding cognitive domain.

According to an embodiment of the present disclosure, a difficulty level may be individually adjusted based on the results of the training of a user, and training having a different difficulty level may be provided for each user.

According to an embodiment of the present disclosure, the results of training may be analyzed. A difficulty level may be generated by raising the difficulty level when next training content is generated if the results of the training are relatively high based on the analyzed results. A difficulty level may be generated by lowering the difficulty level when next training content is generated when the results of the training are relatively low based on the analyzed results.

According to an embodiment of the present disclosure, a training game may be provided by raising the difficulty level of cognitive reinforcement training content to be generated next to a higher level when the results of the training of a user is A, maintaining the difficulty level of cognitive reinforcement training content to be generated next when the results of the training of a user is B, lowering the difficulty level of cognitive reinforcement training content to be generated next to a lower level when the results of the training of a user is C, raising the difficulty level of cognitive reinforcement training content to be generated next to a higher level when the results of the training of a user is a pass, and maintaining or lowering the difficulty level of cognitive reinforcement training content to be generated next to a lower level when the results of the training of a user is a fail.

According to an embodiment of the present disclosure, user personal information including information on the name, gender, age, and training start date of a user may be provided.

According to an embodiment of the present disclosure, the user personal information may be information generated by using information on the name, gender, age, and training start date of a user that are received from the user, but personal information of a user that is necessary for training may be used without limit.

According to an embodiment of the present disclosure, present training situation information including a training attendance, the number of training absences, a training completion number, and the results of training for each area may be provided.

According to an embodiment of the present disclosure, the present training situation information may mean information that has been arranged in a time series by collecting the results of training that has been performed by a user. Any data that has been arranged in a time series with respect to a specific training item, in addition to the training attendance, the number of training absences, the training completion number, and the results of training for each area, may be used as the present training situation information without limit.

According to an embodiment of the present disclosure, a training progress based on the results of training up to now from the first training date of a user may be provided.

According to an embodiment of the present disclosure, the training progress may be provided to a user in a graph form, but the present disclosure is not limited thereto. Any form that may show a time-series progress may be used without limit.

A reward is provided to the user based on the results of the training and the training attendance rate and training completion rate of the user. A personal memory remembrance question generated based on the user personal information and text input by the user is provided (S50).

According to an embodiment of the present disclosure, the reward may be provided to a user based on the results of the training of the user and a training attendance rate and training completion rate of the user. A personal memory remembrance question that is generated based on user personal information and text input by the user may be provided to the user.

According to an embodiment of the present disclosure, the reward may mean compensation capable of inspiring the motivation of a user, such as a point, a stamp, appellation, or a gift, when a training number, a training participation rate, a training achievement rate, or a training mark is equal to or greater than a predetermined reference, but any reward may be used without limit if the reward can be accepted by a user as a compensatory meaning.

According to an embodiment of the present disclosure, the personal memory remembrance question may mean a question about a personal item for the remembrance of memory and the establishment of ties for a user, and may include a user personal item question.

According to an embodiment of the present disclosure, after the present reward information is provided to the user, the induction of remembrance and the building of an alliance may be performed through a user personal item question including at least one of questions about thoughts, feelings, experiences, wishes, expectations, family matters, hobbies, religion, friendship, and his or her advantages and disadvantages.

According to an embodiment of the present disclosure, in order to perform the induction of remembrance and the building of an alliance, at least one of questions about thoughts, feelings, experiences, wishes, expectations, family matters, hobbies, religion, friendship, and his or her advantages and disadvantages may be included in a personal item question. Question items are not limited to the above examples.

According to an embodiment of the present disclosure, a reward having a stamp or point form may be provided to a user by incorporating at least one of items, including a training participation rate of the user, the results of execution of habit formation content of the user, and the results of execution of cognitive reinforcement training of the user. Present reward information of the provided reward may be provided to the user.

An embodiment of the present disclosure is not implemented through only the aforementioned apparatus and/or method. The embodiments of the present disclosure have been described in detail, but the scope of rights of the present disclosure is not limited thereto. A variety of modifications and changes using the basic concept of the present disclosure defined in the appended claims are also included in the scope of rights of the present disclosure.

1. A digital cognitive reinforcement training apparatus for reinforcing cognitive reservability, comprising:
an interactive habit formation content provision unit configured to generate habit formation content comprising preemptive sentence content for providing mild cognitive impaired (MCI) and dementia-associated information and habit information that are collected through queries and answer sentence content suitable for context by analyzing text input by a user and to provide the generated habit formation content;
a cognitive reinforcement training content provision unit configured to generate cognitive reinforcement training content comprising cognitive reinforcement training information for a plurality of cognitive domains and to provide the cognitive reinforcement training content to the user;
a training result provision unit configured to calculate results of training of the user for the provided training content and to provide the results of the training and a present training situation to the user;
a training result feedback unit configured to derive training content difficulty level adjustment information and preferred training content information by analyzing the results of the training, to construct a personalized training content set so that an area that the user lacks of is able to be supplemented based on the derived information, and to incorporate the personalized training content set into next training of the user; and
an interactive compensation provision unit configured to provide a reward to the user based on the results of the training and a training attendance rate and training completion rate of the user and to provide a personal memory remembrance question generated based on personal information of the user and text input by the user.

2. The digital cognitive reinforcement training apparatus of claim 1, wherein:
user basic information comprises at least one of a user name, a date of birth, gender, a user identification code, personal exercise habits, and personal eating habits, and
the interactive habit formation content provision unit collects the basic information of the user in a query form.

3. The digital cognitive reinforcement training apparatus of claim 1, wherein the interactive habit formation content provision unit
periodically provides the user with the habit formation content that is included in a habit list of exercise and eating habits that help a development of cognitive reservability, and
regularly provides a user task to the user based on habits selected by the user from the habit list.

4. The digital cognitive reinforcement training apparatus of claim 3, wherein the interactive habit formation content provision unit
checks whether the provided user task has been performed,
generates a compliment or warning sentence content when frequency of the execution is greater than or less than a preset reference range, and
provides the compliment or warning sentence content to the user.

5. The digital cognitive reinforcement training apparatus of claim 1, wherein the cognitive reinforcement training content provision unit provides the user with cognitive reinforcement training content having a game form, an execution of which needs to be completed within an allowed time every cycle that is input by the user or that is randomly preset, as a set unit.

6. The digital cognitive reinforcement training apparatus of claim 5, wherein:
the cognitive reinforcement training content having the game form comprises cognitive domain information that is a training target and a game tutorial, and
the cognitive reinforcement training content provision unit provides the user with the cognitive domain information, the game tutorial, and game content that is preset within a limited time or randomly generated when the user performs a game.

7. The digital cognitive reinforcement training apparatus of claim 4, wherein the cognitive reinforcement training content provision unit randomly provides the cognitive reinforcement training content generated based on a plurality of cognitive domains, respectively, or provides the cognitive reinforcement training content for an area that is preferred by the user or that requires intensive training, based on a selection of the user or the results of the training of the user.

8. The digital cognitive reinforcement training apparatus of claim 1, wherein the training result feedback unit
collects the results of the training of the cognitive reinforcement training content performed by the user,
generates training result information comprising at least one of a training participation rate, a training achievement rate, results of training for each area by analyzing the collected results of the training, and
provides the training result information to the user.

9. The digital cognitive reinforcement training apparatus of claim 1, wherein the cognitive reinforcement training content provision unit comprises:
a memory training content generation unit configured to generate, in a game form, content comprising training to remind specific information;
a language ability training content generation unit configured to generate, in a game form, content comprising association or analogy training performed by using a sentence or a word;
an execution ability training content generation unit configured to generate, in a game form, training to determine an order of things over time;
an attention concentration training content generation unit configured to generate, in a game form, content comprising training to concentrate attention;
a calculation ability training content generation unit configured to generate, in a game form, training to improve a calculation ability by providing a four fundamental arithmetic operations problem; and
a visual perception training content generation unit configured to generate, in a game form, training to improve a visual perception ability through training to expect a shape that is seen depending on a point of view of a specific object.

10. The digital cognitive reinforcement training apparatus of claim 1, wherein the interactive compensation provision unit
provides the user with a reward having a stamp or point form by incorporating at least one of items comprising a training participation rate of the user, results of execution of the habit formation content, and results of execution of the cognitive reinforcement training, and
provides the user with present reward information of the provided reward.

11. The digital cognitive reinforcement training apparatus of claim 10, wherein after providing the user with the present reward information, the interactive compensation provision unit performs an induction of remembrance and a building of an alliance through a user personal item question comprising at least one of questions about thoughts, feelings, experiences, wishes, expectations, family matters, hobbies, religion, friendship, and his or her advantages and disadvantages.

12. The digital cognitive reinforcement training apparatus of claim 9, wherein the memory training content generation unit
selects one of games comprising “memorizing poems”, “memorizing national flags by country”, “memorizing local names”, a common sense quizzes, and “where to and what to eat” as the training to remind specific information, and
generates the selected game in the game form.

13. The digital cognitive reinforcement training apparatus of claim 9, wherein the language ability training content generation unit
selects one of games comprising “a four-character idiom”, “guess a first letter”, “a rearrangement of words”, and “please guess a name” as the association or analogy training that is performed by using the sentence or word, and
generates the selected game in the game form.

14. The digital cognitive reinforcement training apparatus of claim 9, wherein the execution ability training content generation unit
selects one of games comprising “let's eat”, “travel plans are fun”, and “ordering” as the training to determine the order of things over time, and
generates the selected game in the game form.

15. The digital cognitive reinforcement training apparatus of claim 9, wherein the attention concentration training content generation unit
selects one of “find words”, “find a same picture”, and a location movement game as the training to concentrate attention, and
generates the selected game in the game form.

16. The digital cognitive reinforcement training apparatus of claim 9, wherein the calculation ability training content generation unit
selects one of a receipt game, an “unlock a password” game, a subtract king game, and a “step by step from a basics” game as the training to improve the calculation ability by providing the four fundamental arithmetic operations problem, and
generates the selected game in the game form.

17. The digital cognitive reinforcement training apparatus of claim 9, wherein the visual perception training content generation unit
selects one of a shape prediction game and a “where was a picture taken” game that expects a figure of a shape that is seen depending on a view of a specific object, and
generates the selected game in the game form.

18. The digital cognitive reinforcement training apparatus of claim 1, wherein the training result feedback unit provides training having a different difficulty level for each user by individually adjusting the difficulty level based on the results of the training of the user.

19. The digital cognitive reinforcement training apparatus of claim 18, wherein the training result feedback unit provides a training game by:
raising the difficulty level of the cognitive reinforcement training content to be generated next to a higher level when the results of the training of the user is A,
maintaining the difficulty level of the cognitive reinforcement training content to be generated next when the results of the training of the user is B,
lowering the difficulty level of the cognitive reinforcement training content to be generated next to a lower level when the results of the training of the user is C,
raising the difficulty level of the cognitive reinforcement training content to be generated next to a higher level when the results of the training of the user is a pass, and
maintaining or lowering the difficulty level of the cognitive reinforcement training content to be generated next to a lower level when the results of the training of the user is a fail.

20. The digital cognitive reinforcement training apparatus of claim 1, wherein the training result provision unit comprises:
a personal information generation unit configured to provide user personal information comprising information on a name, gender, age, and training start date of the user;
a present training situation information provision unit configured to provide present training situation information comprising a training attendance, a number of training absences, a training completion number, results of training for each area; and
an area-based training result progress provision unit configured to provide a training progress based on results of the training up to now from a first training date of the user,
wherein the training result provision unit periodically provides a registered third-party terminal with information on the results of the training of the user by using at least one of methods comprising SMS, a website, e-mail, and a messenger message with a consent of the user, and
the interactive habit formation content provision unit identifies a user cognitive state comprising an intention of the user based on text input by the user, generates a natural query sentence suitable for the context as interactive habit formation content based on the identified user cognitive state, and provides the natural query sentence to the user.

21-26. (canceled)
